(19) United States
(12) Patent Application Publication
(10) Pub. No.: US 2011/0124523 A1
(43) Pub. Date: May 26, 2011

(54) High Throughput Method for Detecting Apoptosis of Embryonic Stages of Metazoan Helminthic Parasites

(75) Inventors: Balachandran Ravindran, Orissa (IN); Alok Das Mohapatra, Orissa (IN)

(73) Assignee: INSTITUTE OF LIFE SCIENCES, Bhubaneswar, Orissa (IN)

(21) Appl. No.: 12/935,165

(22) PCT Filed: Nov. 12, 2009
(86) PCT No.: PCT/IN09/00638

§ 371 Date: Sep. 28, 2010

(30) Foreign Application Priority Data
Sep. 11, 2009 (IN) 1145/KOL/090

(51) Int. Cl. C40B 30/06; C12Q 1/18; C12Q 1/37

(52) U.S. Cl. 506/10; 435/32; 435/23

(57) ABSTRACT

A method for detecting apoptosis of embryonic stages of parasitic helminthes. The method comprises isolating of intra uterine embryonic stages from an adult female parasite. The embryonic stages are cultured in vitro and treated. The said embryonic stages are subjected to flow cytometric analysis. An assay for apoptosis is performed being capable of high throughput screening and identification of compounds having apoptogenic activity towards the embryonic stages of helminthic parasites.

### FIELD OF INVENTION

This invention relates to a flow cytometry based, assay system for detecting apoptosis of embryonic stages of parasitic helminthes and high throughput screening of apoptogenic antihelminthic compounds.

### BACKGROUND OF THE INVENTION

Parasitic helminthes cause chronic diseases with severe morbidity in both humans and animals and broadly include two groups i.e. intestinal-geo helminthes like Ascaris, Trichuris, Hook worms and tissue invasive-systemic helminthes like. Filarial and Schistosome parasites etc. Diseases caused by these helminthic parasites have unusually high incidence in developing countries at present e.g. Ascariasis (807 million), Trichuriasis (604 million), Hook woi in infections (574 million), Schistosomiasis (207 million), Lymphatic filariasis (120 million) and Onchocerciasis (37 million) etc. (Hotez, 2008, WHO 2006). These diseases continue to pose serious challenges to the economic welfare and public health in most of the developing countries. In the absence of any preventive vaccine, chemotherapy remains the mainstay for treatment of diseases caused by these metazoan helminthic parasites. Beginning with the widespread use of DEC for the treatment of Lymphatic Filariasis (LF) in China during the 1970s, the mass treatment of human population with antihelminthic drugs known as mass drug administration (MDA), has been a major approach for controlling human helminthiases in developing countries (Hotez, et al., 2007). However, the success of MDA programs is marred by several factors including lack of any effective adulticidal drugs for the parasitic worms (Hotez et al., 2008), limited effect of the existing drugs on the embryogenesis of helminthic parasites (Awadzi et al., 1995, Plaisier et al., 1991), adverse side reactions associated with the drug of choice for human filariasis, Diethylcarbamazine (DEC) (Haarbrink et al., 1999), possibility of emergence of drug resistant parasites coupled with the lack of robust biomarkers for detection of resistance of helminthic parasites to the mainstay drugs of MDA programs (Hotez et al., 2008) etc. The problem is further compounded by the chances of rapid reinfection of hosts by these parasites, post-treatment (Hotez et al., 2008, Loukas et al., 2005). Further, in case of filarial infections the existing drugs like DEC and Ivermectin acts rapidly to reduce the number of circulating microfilariae for only a few months after which microfilariae reappear at levels of 20% or more of pretreatment numbers within a year (Awadzi et al., 1995). This microfilarial density is presumed to be sufficient for continuation of transmission of the parasite (Alley et al., 2001). The reason for this rather limited effect of chemotherapy in filarial infections is that it does not kill the long lived adult worms and that its embryocidal activity seems to be mainly restricted to the final embryonic stages i.e. microfilariae, leaving early embryogenesis intact (Plaisier et al., 1991). Studies using Albendazole have also revealed very little effect on embryogenesis of filarial nematodes (Awadzi et al., 1995). In such a scenario, worldwide elimination of diseases caused by metazoan helminthic parasites including lymphatic filariasis may remain a distant goal, not achievable without development of better drugs. Hence, new approaches for chemotherapy with well defined mode of action are an acute necessity for controlling the rising helminthic infections in human and animal communities (Hotez et al., 2008, Hoerauf et al., 2002; Melrose, 2003).

Being obligate parasites with no intermediate animal hosts, persistence of these helminthic parasites in their mammalian hosts becomes central to their survival. This is materialized by release of a large number of embryonic stages called larval stage-1/L-1/microfilariae into peripheral circulation of the infected hosts (in case of systemic helminthic parasites) or fertilized eggs in to the soil/water (in case of parasitic geo-helminthes) by adult female worms which are subsequently transmitted into new susceptible hosts by insect vectors in case of former and through contaminated food and water in case of later. Thus, successful embryogenesis in adult female parasites is a critical rate limiting step required for survival and propagation of the parasitic worms in host communities. Hence, agents that can induce apoptosis in the fertilized eggs or embryonic stages of helminthic parasites can be expected to have immense potential for elimination of such pathogens from human and animal communities by blocking their embryokenesis and subsequent transmission. However, currently there are no established reports available in literature regarding apoptosis in the embryonic stages as well as drugs blocking embryogenesis in parasitic worms. Further, microscopy, the only tool used to score apoptosis in the free living soil nematode C. elegans permits analysis of only limited number of apoptotic features and cells/embryonic stages. In this context the current study demonstrating high throughput assays for apoptosis in fixed as well as live embryonic stages of a metazoan helminthic parasite filarial parasite S. digitata is a quantum improvement in the study of embryogenesis of parasitic helminthes and can be used for high of anti-helminthic compounds having apoptogenicity towards embryonic stages of parasitic worms.

### OBJECTS OF THE INVENTION

An object of this invention is to propose assays for detecting apoptosis of embryonic stages of parasitic helminthes;

Another object of this invention is to demonstrate the possibility of high throughput screening and identification of compounds having apoptotogenic and antihelminthic activity against the embryonic stages of a metazoan helminthic parasite using three different agents namely Plumbagin, H₂O₂ and Staurosporine;

Still another object of this invention is to propose assays to detect and quantify multiple conserved features of apoptosis such as externalization of phosphatidyl serine.

Yet another object of this invention is to propose assays to detect and quantify multiple conserved features of apoptosis such as depolarization of mitochondria;

Further, object of this invention is to propose assays to detect and quantify multiple conserved features of apoptosis such as enhanced cytosolic presence of cytochrome-c;

Still further object of this invention is to propose assays to detect and quantify multiple conserved features of apoptosis such as increased intra cellular expression of nematode apoptosis related proteins CED-3, CED-4 & CDE-9;

Yet further object of this invention is to propose assays to detect and quantify multiple conserved features of apoptosis such as activation of caspase family of cysteine proteinases;

Still further object of this invention is to propose assays to detect and quantify multiple conserved features of apoptosis such as cleavage of intracellular caspase substrate PARP;

Another object of this invention is to propose assays to detect and quantify multiple conserved features of apoptosis such as fragmentation of chromosomal DNA and formation of sub-diploid nuclei in the embryonic stages of filarial parasite S. digitata using 3 known apoptosis inducing agents namely Plumbagin, H₂O₂ and Staurosporine.

### BRIEF DESCRIPTION OF THE INVENTION

According to this invention there is provided a method for detecting apoptosis of embryonic stages of helminthes comprising:

isolating Intra Uterine embryonic stages from adult female parasite; culturing in vitro and treating said embryonic stages;
subjecting the said embryonic stages to the step of flow cytometric analysis;
developing assays for apoptosis in the embryonic stages
and screening of three compounds for their apoptogenicity against embryonic stages of helminthic parasite S. digitata.

### DETAILED DESCRIPTION OF THE INVENTION

In the present invention we report standardization and evaluation of 10 novel quantitative flow cytometry based assays to detect and quantify multiple conserved features of apoptosis such as externalization of phosphatidyl serine, mitochondrial depolarization, enhanced cytosolic presence of cytochrome c, increased expression of nematode apoptosis related proteins CED-3, CED-4 and CED-9, activation of caspase family of cysteine proteinases, cleavage of intracellular caspase substrate PARP, fragmentation of chromosomal DNA and formation of sub-diploid nuclei etc. in the embryonic stages of filarial parasite S. digitata using three known apoptosis inducing agents (e.g. Plumbagin, H₂O₂ and Staurosporine). These assays constitute the first ever report on development and evaluation of flow cytometery based assays for apoptosis of embryonic stages in helminthic parasites. These assays also offer opportunities for development of automated high throughput screening assays for identifying apoptosis inducing agents/drugs to block embryogenesis in parasitic helminthes which can potentially affect their transmission and survival in host communities.

Apoptosis of embryonic stages of parasitic helminthes has not been reported earlier in literature primarily due to lack of sensitive assay system. The current invention describes 10 different flow cytometry based assays for detecting and quantifying apoptosis in the embryonic stages of a filarial nematode S. digitata.
Method-1. Isolation of Intra Uterine Embryonic stages from Adult Female parasites.
Method-2. In-vitro culture and Treatment of Embryonic stages.
Method-3. Flow cytometric analysis of the Embryonic stages.
Method-4. Assays for Apoptosis in the Embryonic stages
(a) Detection of externalization of Phosphatidyl Serine
(b) Analysis of Mitochondrial depolarization
(c) Detection of increased intra cellular expression of CED-3 protein—a member of the caspase family of cystiene proteinases.
(d) Detection of increased intra cellular expression of CED-4 protein—a homologue of mammalian Apaf-1
(e) Detection of increased intra cellular expression of CED-9 a homologue of mammalian Bcl₂ protein.
(f) Demonstration of enhanced cytosolic presence of cytochrome-c in the cytoplasm.
(g) Demonstration of intra cellular activation of caspase-3 like cysteine proteinases.
(h) Demonstration of intra cellular cleavage of caspase substrate PARP.
(i) Detection of Fragmentation of Chromosomal DNA.
(j) Demonstration of the presence of Sub-diploid Nuclei.

Method-1—Isolation of Intra Uterine Embryonic Stages from Adult Female Parasite.

Adult female filarial worms Setaria digitata were collected from the peritoneum of cattle, slaughtered at a nearby abattoir in sterile Hanks Balanced Salt Solution (HBSS) medium (Sigma H 2387-1L) and transported to the laboratory in a sterile container. The medium containing 1% glucose (Sigma G 7528), Penicillin 100 units/ml, Streptomycin 100 μg/ml (Sigma P 4333). And Amphotericin-B 0.25 μg/ml (Sigma A 2942) was buffered with NaHCO₃ (Sigma S 5761). About 5 to 7 worms were taken in a petridish, washed three times in medium, dissected into small pieces in 10 ml of medium under sterile conditions and incubated at 37° C. for 30 minutes to allow the release of embryonic stages (eggs and microfilariae) into the medium. The embryonic stages were harvested into sterile 15 ml. centrifuge tubes and washed three times by centrifuging at 300 g for 10 minutes each with medium and the final pellet was suspended in 1 ml. of RPMI-1640 medium (Sigma R 8005) supplemented with 10% FBS (Sigma F 2442)

Method-2—In-Vitro Culture and Treatment of Embryonic Stages.

The number of embryonic stages of S. digitata in the above preparation were counted using light microscope, and the suspension was taken in RPMI-1640 medium containing 10% FBS at 1×10⁵ embryonic stages/ml. One ml. each was dispensed into individual wells of a 24 well tissue culture plate. One set of wells was taken as untreated control containing only medium with 10% FBS. Other sets of wells were subjected to treatment with agents like H₂O₂ (Sigma H 1009) or Plumbagin (Sigma P 7262) at a concentration ranging from 10-100 μm while treatment with Staurosporine (Sigma S 3939) was performed at a concentration range of 0.5-5.0 μM for 24 hr. at 37° C. in a 5% CO₂ incubator. Motility of the Mf was scored under an inverted microscope.

Method-3—Flow Cytometric Analysis of the Embryonic Stages

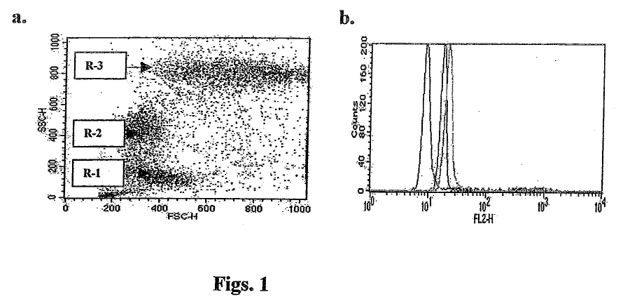

After appropriate treatment and incubation, each set of the cultured embryonic stages of S. digitata were harvested from the 24 well tissue culture plate separately in 15 ml. sterile centrifuge tubes, washed with PBS twice and stained as per requirement following manufacturer's instructions as described below. This was followed by two times washing with PBS and final suspension of the stained embryonic stages was taken in 0.5 ml of sheath fluid (BD Biosciences 342003) before their acquisition in the flow cytometer (BD FACS Calibur, Becten and Dickinson, USA). Analysis was performed on 10,000-acquired events depicted as Dot Plots and Histogram Plots using Cellquest Pro soft ware. All plots were representative of at least 3 experiments. The distinctly clustered populations obtained consistently in the Dot plots for the intra uterine embryonic stages of S. digitata (FIG. 1 a) were gated for Mfs (R-1) and eggs (R-2 and R-3) and analyzed for different assays for apoptosis as described below.

Method-4. Assays for Apoptosis in the Embryonic Stages

(a)—Detection of Externalization of Phosphatidyl Serine.

The externalization of phosphatidyl serine was detected by staining the embryonic stages with Annexin-V-PE (BD Biosciences 556421) as per the instructions of the manufacturer. Briefly, the embryonic stages of S. digitata were subjected to various treatments in culture for 24 hrs. After the said period of incubation the embryonic stages were harvested, washed with ice cold PBS twice and resuspended in 100 μl of 1× Annexin-V binding buffer (BD Biosciences 556454) at 1×10⁵ embryonic stages per ml. to which 5 μl of Annexin-V-PE was added. After 15 minutes of Incubation in dark at room temperature another 400 μl of 1× Annexin-V binding buffer was added to the above suspension. The resulting 500 μl suspension of Annexin-V-PE stained embryonic stages was then acquired and analyzed by flow cytometer.

(b) Analysis of Mitochondrial Depolarization.

The mitochondrial membrane potential (ΔΨm) was measured using JC-1 probe (a cataionic lipophilic fluorochrome that accumulates in mitochondria in proportion to mitochondrial membrane potential) supplied in mitoscreen JC-1 kit (BD Biosciences 551302). Briefly a suspension of in-vitro cultured embryonic stages at 1×10⁵ embryonic stages/ml. was mixed with 0.5 ml of freshly prepared JC-1 working solution for 10-15 minutes at 37° C. in a CO₂ incubator followed by two times washing and final suspension of the embryonic stages in 1× assay buffer (supplied with the kit) before acquisition and analysis by flow cytometry. Reduction of fluorescence intensity of the mitoscreen JC-1 stained embryonic stages was taken as the degree of depolarization of mitochondria.

(c) Detection of Increased Intra Cellular Expression of CED-3 Protein.

After in vitro culture for 24 hours with apoptosis inducing reagents the embryonic stages were harvested in 15 ml, sterile centrifuge tubes and fixed for 60 minutes at 0° C. in 1% paraformaldehyde (Sigma P 6148) in PBS. The suspension was washed first with PBS and then with 1× permeabilisation buffer (eBioscience 00-8333-56) and finally resuspended in 100 μl of 1× permeabilisation buffer at 1×10⁵ embryonic stages per ml. The intra cellular expression of CED-3 was scored by incubation of the fixed and permeabilised embryonic stages with primary goat antibodies—anti CED-3 (Santacruz sc-9192) followed by Probing with PE conjugated secondary anti goat IgG-PE (Santacruz sc-3747) antibodies. Incubation of both primary and secondary antibodies (both 1/100 diluted) was done successively in 1× permeabilisation buffer (e Bioscience 00-8333-56) for 1 hr and 45 minutes respectively. The suspension of stained embryonic stages was then washed with PBS twice and finally reading was taken by flow cytometry.

(d) Detection of Increased Intra Cellular Expression of CED-4 Protein.

After in vitro culture for 24 hours with apoptosis inducing reagents the embryonic stages were harvested in 15 ml. sterile centrifuge tubes and fixed for 60 minutes at 0° C. in 1% paraformaldehyde (Sigma P 6148) in PBS. The suspension was washed first with PBS and then with 1× permeabilisation buffer (eBioscience 00-8333-56) and finally resuspended in 100 μl of 1× permeabilisation buffer at 1×10⁵ embryonic stages per ml. The intracellular expression of CED-4 was scored by incubation of the fixed and permeabilised embryonic stages with primary goat antibodies—anti CED-4 (Santacruz sc-9193) followed by probing with PE conjugated secondary anti goat IgG-PE (Santacruz sc-3747) antibodies. Incubation of both primary and secondary antibodies (both 1/100 diluted) was done successively in 1× permeabilisation buffer (e Bioscience 00-8333-56) for 1 hr and 45 minutes respectively. The suspension of stained embryonic stages was then washed with PBS twice and finally reading was taken by flow cytometry or confocal microscopy.

(e) Detection of Increased Intra Cellular Expression of CED-9 Protein.

After in vitro culture for 24 hours with apoptosis inducing reagents the embryonic stages were harvested in 15 ml. sterile centrifuge tubes and fixed for 60 minutes at 0° C. in 1% paraformaldehyde (Sigma P 6148) in PBS. The suspension was washed first with PBS and then with 1× permeabilisation buffer (eBioscience 00-8333-56) and finally resuspended in 100 μl of 1× permeabilisation buffer at 1×10⁵ embryonic stages per ml. The intracellular expression of CED-9 was scored by incubation of the fixed and permeabilised embryonic stages with primary goat antibodies—anti CED-9 (Santacruz sc-9202) followed by probing with PE conjugated secondary anti goat IgG-PE (Santacruz sc-3747) antibodies. Incubation of both primary and secondary antibodies (both 1/100 diluted) was done successively in 1× permeabilisation buffer (e Bioscience 00-8333-56) for 1 hr and 45 minutes respectively. The suspension of stained embryonic stages was then washed with PBS twice and finally reading was taken by flow cytometry or confocal microscopy.

(f) Demonstration of Enhanced Presence of Cytochrome-c in the Cytoplasm.

After in vitro culture for 24 hours with apoptosis inducing reagents the embryonic stages were harvested in 15 ml. sterile centrifuge tubes and fixed for 60 minutes at 0° C. in 1% paraformaldehyde (Sigma P 6148) in PBS. The suspension was washed first with PBS and then with 1× permeabilisation buffer (eBioscience 00-8333-56) and finally resuspended in 100 μl of 1× permeabilisation buffer at 1×10⁵ embryonic stages per ml. This suspension was then incubated for 45 minutes with 20 μl of anti cytochrome c-FITC (eBioscience 11-6601-82). This was followed by washing with PBS twice and analysis by Flow cytometer.

(g) Demonstration of Intra Cellular Activation of Caspase-3 Like Cystiene Proteinases.

After in vitro culture for 24 hours with apoptosis inducing reagents the embryonic stages were harvested in 15 ml. sterile centrifuge tubes and fixed for 60 minutes at 0° C. in 1% paraformaldehyde (Sigma P 6148) in PBS. The suspension was washed first with PBS and then with 1× permeabilisation buffer (eBioscience 00-8333-56) and finally resuspended in 100 μl of 1× permeabilisation buffer at 1×10⁵ embryonic stages per ml. This suspension was then incubated for 45 minutes with 20 μl of anti active caspase-3-PE (BD Biosciences 559762). This was followed by washing with PBS twice and analysis by Flow cytometer.

(h) Demonstration of Intra Cellular Cleavage of Caspase Substrate PARR

After in vitro culture for 24 hours with apoptosis inducing reagents the embryonic stages were harvested in 15 ml. sterile centrifuge tubes and fixed for 60 minutes at 0° C. in 1% paraformaldehyde (Sigma P 6148) in PBS. The suspension was washed first with PBS and then with 1× permeabilisation buffer (eBioscience 00-8333-56) and finally resuspended in 100 μl of 1× permeabilisation buffer at 1×10⁵ embryonic stages per ml. This suspension was then incubated for 45 minutes with 20 μl of anti cleaved PARP-PE (BD Biosciences 552933). This was followed by washing with PBS twice and analysis by Flow cytometer.

(i) Detection of Fragmentation of Chromosomal DNA.

Presence of fragmentation of chromosomal DNA was detected by TUNEL staining. Briefly, the treated embryonic stages were harvested, fixed with 1% Para formaldehyde followed by washing with PBS and stored in 70% ethanol at −20° C. After 18 hrs they were washed and subjected to TUNEL staining at 1×10⁵ embryonic stages per ml. of suspension using the APO-Direct apoptosis detection kit (BD Biosciences 556381) and analyzed by flow cytometer.

(j) Demonstration of the Presence of Sub-Diploid Nuclei.

Presence of sub-diploid nuclei was detected by PI/RNase staining. Briefly, the treated embryonic stages were harvested, fixed with 1% Para formaldehyde followed by washing with PBS and stored in 70% ethanol at −20° C. After 18 hrs they were washed and subjected to PI/RNase staining at 1×10⁵ embryonic stages per ml. of suspension using the APO-Direct apoptosis detection kit (BD Biosciences 556381) and analyzed by flow cytometer.

### Results

Initially externalization of phosphatidyl serine, mitochondrial depolarization, cytosolic presence of cytochrome c, activation of caspase family of cysteine proteases and break down of caspase substrate-PARP, fragmentation of chromosomal DNA and formation of sub-diploid nuclei etc. were analyzed in the embryonic stages of the pathogenic filarial nematode S. digitata. Apoptosis induction was further confirmed by quantifying apoptosis related nematode proteins viz. CED-3, CED-4 and CED-9. Three compounds namely Plumbagin, H2O2 and Staurosporine were screened for their apoptogenicity against embryonic stages of filarial parasite S. digitata.

### Externalization of Phosphatidyl Serine

Phosphatidyl serine is a membrane phospholipid, usually confined to the inner bilayer of the membrane. In mammalian cells the inner bilayer phospholipid has been demonstrated to get translocated to the outer bilayer during apoptosis (Elmore et al., 2007). To assess if such externalization of phosphatidyl serine takes place in nematodes, embryonic stages of S. digitata were subjected to Annexin-V-PE staining and scored by flow cytometry. A dose dependent externalization of Phosphatidyl serine was observed in Microfilariae (Mfs) (FIG. 1b) as well as in eggs (Table-1) upon treatment with Plumbagin and H₂O₂ while Staurosporine did not mediate such effects on Phosphatidyl serine externalization. The externalization of PS upon treatment was relatively more evident in treated egg stages when compared to Microfilariae (Table-1).

### Disruption of Mitochondrial Trans Membrane Potential

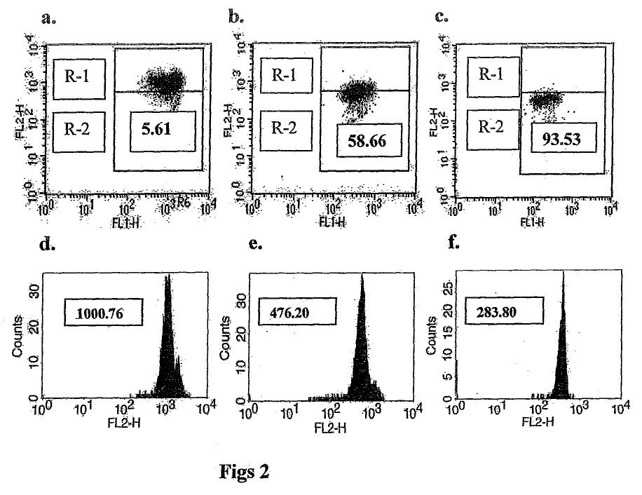

Mitochondria is known to be one of the important regulators of metazoan apoptosis (Arnoult et al., 2002) as the signals generated by various death promoting agents converge in mitochondria leading to reduction in trans membrane potential (ΔΨm) in several models of apoptosis (Zamzami et al., 2001). Embryonic stages of S. digitata were subjected to mitoscreen JC-1 staining. A differential and dose dependent induction of mitochondrial depolarization was observed both in Mfs (FIG. 2) and eggs (Table-1) on treatment with Plumbagin and H₂O₂ but not with Staurosporine. Mf stages displayed significantly more mitochondrial depolarization upon treatment in comparison to eggs (Table-1).

### Intra Cellular Profile of Proteins Related to Apoptosis

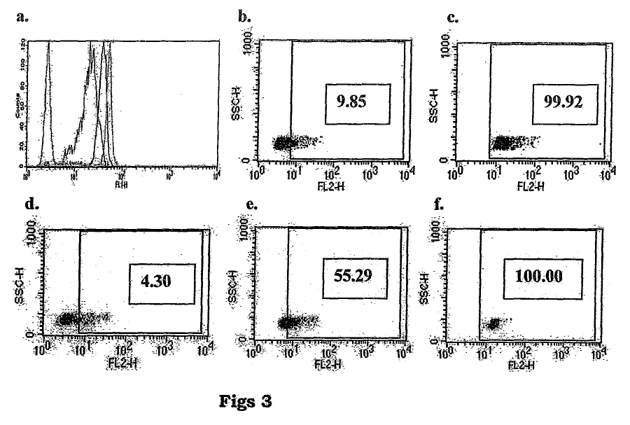

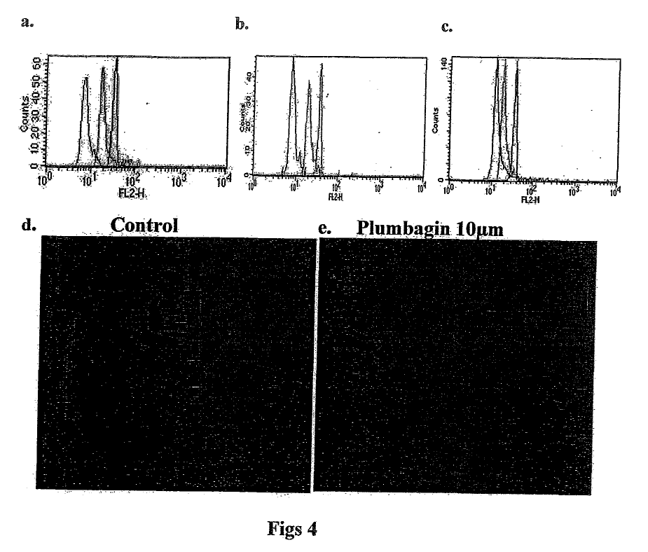

Intra cellular staining of the fixed embryonic stages of S. digitata with antibodies to cytochrome c, active caspase-3 and cleaved PARP revealed enhanced cytosolic presence of cytochrome c (FIG. 3a), activation of mammalian caspase-3 like proteases (FIG. 3b, c) and cleavage of the substrate of caspase family of proteases viz. PARP (FIG. 3d, e, f) in Mf as well as in treated eggs (Table-1) undergoing apoptosis. Studies in the free living soil nematode C. elegans had identified three nematode specific proteins such as CED-3, CED-4 and CED-9 associated with the process of apoptosis. Homolog of the three proteins have been found in genomes of all animals and were shown to be involved in apoptosis in all systems studied so far (Zamsek et al., 2007). The status of these three proteins CED-3, CED-4 and CED-9 was investigated in embryonic stages of pathogenic nematode S. digitata and increased intracellular expression of all the three proteins was observed in Mfs (FIG. 4 a,b,c) as well as in eggs (Table-1). Further, shedding of CED-4 from mitochondria could also be demonstrated (FIG. 4 d, e). The degree of expression of the three proteins was more prominent in Mfs in comparison to egg stages. However, the proportionate increase of proapoptotic proteins CED-3 and CED-4 was found to be more compared to anti apoptotic protein CED-9 (Table-1) which satisfies the biological condition required for induction of apoptosis.

### Fragmentation of Chromosomal DNA and Sub-Diploid Nuclei.

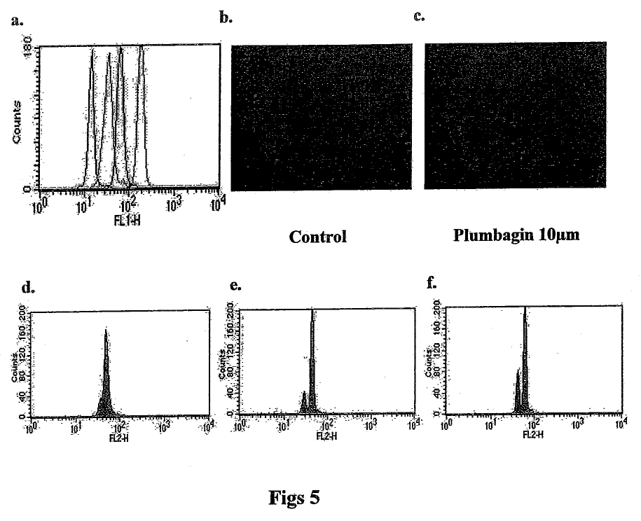

Demonstration of caspase family of cysteine proteinases as described above was followed by investigations on analysis of nuclear features of apoptosis i.e. fragmentation of chromosomal DNA in embryonic stages of S. digitata by TUNEL assay and PI (propidium iodide)/RNase staining. TUNEL assay is generally considered to be a sensitive and confirmatory assay to detect chromosomal DNA fragmentation in apoptotic cells and the results are shown in FIG. 5 a-c. A dose dependent induction of DNA fragmentation in Mf stages (FIG. 5a) and Eggs (Table-1) on treatment with Plumbagin and H₂O₂ but not with Staurosporine (at indicated concentrations) was observed. Moreover, Plumbagin was found to be more efficient than H₂O₂ in induction of apoptosis. PI is a fluorogenic compound that binds stoichiometrically to nucleic acids (Ormerod et al., 2002) so that fluorescence emission from PI stained cells is proportional to the DNA content of the cells (provided RNA is removed by RNase treatment). When apoptotic cells are stained with PI/RNase and analyzed by flow cytometery a distinct hypo-diploid peak (representing the apoptotic nuclei) adjacent to the normal diploid peak (representing healthy nuclei) is displayed in a Histogram plot (Riccardi et al., 2006). In the present study flow cytometric analysis after PI/RNase staining yielded similar results (FIG. 5d-f)

TABLE 1
GMI (Geometric Mean Intensity) of Fluorescence for each of the 9 assays (except
PI/RNase staining for sub-diploid nuclei out of the 10 assays described here)

| | | Microfilariae | | | | Eggs | | | |
|---|---|---|---|---|---|---|---|---|---|
| Sl. | | | Staurosporine | H₂O₂ | Plumbagin | | Staurosporine | H₂O₂ | Plumbagin |
| No | Assay* | Control | (1 μM) | (10 μM) | (10 μM) | Control | (1 μM) | (10 μM) | (10 μM) |
| 1. | Annexin-V- | 7.39 | 8.03 | 11.02 | 12.52 | 27.65 | 28.53 | 33.55 | 85.22 |
| | PE Staining | 8.24 | 8.52 | 10.35 | 13.56 | 29.53 | 30.63 | 35.75 | 89.36 |
| | | 6.95 | 7.24 | 9.75 | 11.95 | 25.27 | 27.59 | 30.95 | 81.25 |
| 2. | CED-3 | 7.95 | 8.33 | 10.99 | 32.75 | 93.95 | 92.71 | 98.25 | 205.26 |
| | Expression | 8.32 | 8.10 | 12.38 | 34.96 | 95.73 | 96.07 | 100.22 | 207.37 |
| | | 7.65 | 7.39 | 9.95 | 31.33 | 92.57 | 90.32 | 97.56 | 200.17 |
| 3. | CED-4 | 8.32 | 9.01 | 15.72 | 35.97 | 94.55 | 95.67 | 99.36 | 209.76 |
| | Expression | 9.56 | 9.92 | 17.33 | 38.27 | 97.62 | 97.28 | 100.32 | 213.57 |
| | | 7.39 | 8.25 | 16.26 | 33.39 | 95.57 | 96.32 | 95.29 | 210.32 |
| 4. | CED-9 | 10.25 | 10.32 | 15.66 | 30.96 | 105.26 | 106.36 | 115.67 | 206.36 |
| | Expression | 12.33 | 9.56 | 17.26 | 33.25 | 111.32 | 109.16 | 123.32 | 202.92 |
| | | 9.56 | 11.02 | 16.13 | 32.27 | 108.56 | 107.34 | 113.26 | 205.16 |
| 5. | Cyt-c | 4.60 | 5.07 | 7.53 | 18.69 | 55.39 | 54.33 | 62.37 | 70.29 |
| | Staining | 5.36 | 6.02 | 9.23 | 20.32 | 60.32 | 61.66 | 67.72 | 76.25 |
| | | 3.62 | 4.20 | 8.14 | 17.56 | 56.37 | 57.27 | 65.39 | 71.32 |
| 6. | PARP | 6.20 | 6.57 | 8.39 | 10.01 | 62.35 | 63.95 | 70.72 | 102.15 |
| | Cleavage | 5.32 | 6.21 | 7.99 | 11.32 | 65.20 | 66.56 | 75.32 | 109.36 |
| | | 4.95 | 5.49 | 8.02 | 9.67 | 69.17 | 70.27 | 78.52 | 110.25 |
| 7. | Caspase-3 | 5.97 | 6.20 | 7.57 | 10.57 | 51.29 | 50.13 | 56.95 | 82.35 |
| | Activation | 6.25 | 6.72 | 9.69 | 11.22 | 55.76 | 54.95 | 61.37 | 87.32 |
| | | 8.56 | 7.95 | 12.34 | 14.16 | 52.39 | 50.97 | 60.20 | 80.16 |
| 8. | TUNEL | 10.32 | 10.67 | 13.62 | 40.65 | 65.69 | 65.77 | 70.85 | 104.33 |
| | Staining | 9.92 | 10.02 | 12.52 | 38.97 | 62.32 | 60.33 | 71.36 | 109.68 |
| | | 11.32 | 12.15 | 14.16 | 45.76 | 70.25 | 71.25 | 72.56 | 105.32 |
| 9. | Mitoscreen- | 900.56 | 895.30 | 785.33 | 435.26 | 1560.98 | 1549.35 | 1225.16 | 970.16 |
| | jc-1 | 1000.76 | 979.20 | 823.57 | 476.20 | 1499.14 | 1500.65 | 1165.12 | 1078.76 |
| | staining** | 950.22 | 930.16 | 750.62 | 398.76 | 1530.67 | 1510.50 | 1295.39 | 897.56 |

*The values in the table represent GMI (Geometric Mean Intensity) of Fluorescence for each of the 9 assays
**Mitochondrial Depolarisation assay by Mitoscreen-JC-1-Staining- expressed in terms of reduction in GMI of fluorescence

1-5. (canceled)

6. A method for detecting apoptosis of embryonic stages of helminthes comprising:
isolating intra uterine embryonic stages from an adult female parasite;
culturing and treating said embryonic stages in vitro;
subjecting said embryonic stages to flow cytometric analysis;
performing an assay for apoptosis in the embryonic stages; and
screening of compounds for apoptogenicity against embryonic stages of helminthic parasite S. digitata.

7. The method according to claim 6, further comprising:
harvesting said embryonic stages in sterile centrifuge tubes;
washing said embryonic stages in a medium;
centrifuging said embryonic stages in said medium thereby forming a final pellet;
suspending said final pellet in said medium, wherein the medium is supplemented with 10% FBS;
counting said embryonic stages under a microscope;
dispensing a first sample containing said embryonic stages into a first well of a sterile tissue culture plate;
dispensing a second sample containing embryonic stages into a second well of said sterile tissue culture plate; and
subjecting said first sample to a treatment agent.

8. The method according to claim 6, further comprising
harvesting said embryonic stages from different sets of wells after said culturing step;
washing said embryonic stages with PBS; and
staining said embryonic stages.

9. The method according to claim 6, wherein the assay for apoptosis in embryonic stages is selected from the group consisting of:
a) detecting externalization of phosphatidyl serine;
b) analyzing mitochondrial depolarization;
c) detecting increase in intracellular expression of CED-3 protein;
d) detecting increase in intracellular expression of CED-4 protein;
e) detecting increase in intracellular expression of CED-9 protein;
f) demonstrating enhanced presence of cytochrome-c in the cytoplasm;
g) demonstrating intracellular activation of caspase-3 like cystiene proteinases;
h) demonstrating intracellular cleavage of caspase substrate PARP;
i) detecting fragmentation of chromosomal DNA; and
j) demonstrating the presence of hypo-diploid nuclei.

10. The method according to claim 6, wherein the assay for apoptosis screens and identifies a broad spectrum of antihelminthic compounds having apoptogenic activity.
